(19) United States
(12) Patent Application Publication
(10) Pub. No.: US 2016/0075983 A1
(43) Pub. Date: Mar. 17, 2016

(54) Mycelium Culture Flask

(71) Applicant: Hunan Xinhui Pharmaceutical Co., LTD, Changsha City (CN)

(72) Inventor: Shujin He, Changsha City (CN)

(21) Appl. No.: 14/854,656

(22) Filed: Sep. 15, 2015

(30) Foreign Application Priority Data
Sep. 17, 2014 (CN) 201420535473.7

(51) Int. Cl. C12M 1/24; C12M 1/00

(52) U.S. Cl. CPC C12M 23/08; C12M 23/38

(57) ABSTRACT

A mycelium culture flask is disclosed in the disclosure. The mycelium culture flask comprises a flask body with an upper opening and a lower opening and two flask caps configured to be snap-fitted with the upper opening and the lower opening respectively, and gaps are provided at the edge of the cap. By using the culture flask of the present disclosure, the culturing effect is better, the culturing period is effectively shortened, the contamination rate is reduced, and the growth and transformation of the mycelium are more complete; also, it is convenient to replace the sponge and clean the flask cap.

### CROSS REFERENCE TO RELATED APPLICATION

This application claims priority to Chinese Patent Application No. 201420535473.7 filed Sep. 17, 2014, the contents of which are incorporated by reference.

### FIELD OF THE INVENTION

The present disclosure relates to a container for culturing mycelium, and in particular to a mycelium culture flask.

### BACKGROUND OF THE INVENTION

A culture flask is often used in the process of culturing mycelium. The culture flask in the prior art is comprised of a flask body and a flask cap which are made of polyethylene materials. The flask body has a top opening and the cap is snap-fitted with the opening so as to close the opening. The flask cap is comprised of an upper cap and a lower cap snap-fitted with each other, a sponge is sandwiched between the upper cap and the lower cap, and a plurality of holes are uniformly distributed around the inner side of the lower cap such that the air can be filtered into the flask through the sponge. Such culture flask is suitable for solid culture of mycelia or cultivation of mushrooms. During the use of the culture flask, putting a culture medium blended evenly into the flask and leaving a small amount of space at the opening, capping, steam sterilizing, inoculating in an inoculation hood, and culturing. However, the disadvantages of the existing culture flasks are as follows. (1) The inoculation can only be made at one end, thus the mycelium grows downward after the inoculation, because of lacking oxygen during the growth, it is difficult for the mycelium to growth downward, and the mycelium grows slowly and even does not grow; when the mycelium grows to about a half, it has to open the cap due to the slow growth rate caused by lack of oxygen in the lower part of the flask, although the mycelium grows downward after the cap is opened, the mycelium is easy to be contaminated by undesired microbes, resulting that the upper part of the flask may be contaminated by undesired microbes while the lower part of the flask has not been sufficiently grown, which makes the mycelium be contaminated seriously, and thus leading to poor quality or low yield; the disadvantages in culturing mycelium with such flask are in that the period of culture is long, the growth and transformation of the mycelium are not complete (the bottom of the flask is not sufficiently grown by the mycelium), the contamination rate is high, the cost is high, and so on. (2) It is difficult to clean the flask cap, and it is difficult to separate the upper cap from the lower cap, and a special tool with specially made tip is required to separate the upper cap from the lower cap, also, it is not easy to replace the sponge sandwiched between the upper cap and the lower cap, and it is very inconvenient to clean the caps, which wastes time and energy, and also it requires high labor costs for replacing the sponge and cleaning the flask caps.

### SUMMARY OF THE DISCLOSURE

The present disclosure aims to overcome the disadvantages in the prior art by providing a mycelium culture flask.

In order to achieve the above object, the present disclosure provides the following technical solutions.

The mycelium culture flask comprises a flask body with an upper opening and a lower opening, and two flask caps configured to be snap-fitted with the upper opening and the lower opening respectively, and gaps are provided at the edge of the flask cap.

Preferably, the flask cap comprises an A cap configured to be snap-fitted with the opening and a B cap configured to be snap-fitted with the A cap; a sponge is sandwiched between the A cap and the B cap; A holes, preferably 4 to 10 A holes, are provided in the face of the A cap; and gaps, preferably 2 to 10 gaps, are provided at the edge of the A cap in contact with the B cap.

Preferably, an inner pad is provided at the neck of the lower opening of the flask body, and B holes, preferably 1 to 5 B holes, are provided in the face of the inner pad. The inner pad engages with an inner edge of the lower opening of the flask body through its protruding pad edge.

The mycelium culture flask of the present disclosure is made of a polyethylene material which is resistant to high temperature and high pressure. Both ends of the culture flask of the present disclosure have openings, thus the inoculation can be achieved at both ends and the mycelium grows towards the middle of the culture flask at a high growth rate. The flask cap of the culture flask is comprised of an A cap and a B cap, wherein the A cap is snap-fitted with the opening, and the B cap is snap-fitted with the A cap; A holes are provided in the face of the A cap, and gaps are provided at the edge of the A cap in contact with the B cap, such that the air passes through the gap and is filtered by the sponge to enter into the culture flask from the hole in the face of the A cap, so as to supply the mycelium in the culture flask with oxygen gas; gaps are provided at the edge of the A cap in contact with the B cap, which facilitate separating the A cap from the B cap (it is easy to disassembly the A cap and the B cap at the gap), easily getting the sponge out, cleaning the A cap and the B cap, putting a new sponge into the dried A cap and B cap, and snap-fitting the A cap with the B cap; providing gaps at the edge of the A cap in contact with the B cap also facilitate exuding extra water in the flask. After the culture flask (containing the culture medium) is steam sterilized, the steam penetrates into the culture flask such that the amount of the water in the culture flask may be increased and there may be extra water remaining at the bottom of the flask, thus the gap facilitates exuding the extra water. An inner pad is provided at the neck of the lower opening of the flask body, on one hand, the inner pad is configured to support the solid culture medium in the culture flask, on the other hand, there is a certain space between the inner pad and the cap. Thus, for the upper opening, a space is left to be used for inoculation (placing the strains), and after the inoculation at the upper opening is completed, the flask is inversed such that the lower opening becomes the upper opening, then getting the inner pad out and inoculating (the space left by the inner pad is used for placing the strains).

The following Table 1 indicates a comparative experiment of the growth and transformation of the mycelia in the culture flask of the present disclosure and in a normal culture flask. As known from the results in Table 1, by using the culture flask of the present disclosure, the culturing effect is better, the culturing period is effectively shortened, the contamination rate is reduced, the growth and transformation of the mycelium are more complete, and the contents of effective components are higher; also, it is convenient to replace the sponge and to clean the flask caps.

TABLE 1

| The normal culture flask | | | | | | The culture flask of the present disclosure | | | | | |
|---|---|---|---|---|---|---|---|---|---|---|---|
| | Contam- | Yield | | Content of effective | | | Contam- | Yield | | Content of effective | |
| | ination | rate | Period | components | | | ination | rate | Period | components | |
| Batch No. | rate (%) | (%) | (day) | Cellulase | Ergosterol | Batch No. | rate (%) | (%) | (day) | Cellulase | Ergosterol |
| 20140421-J | 17.5 | 82.5 | 54 | 137 u/g | 0.12% | 20140712-J | 2.0 | 98.0 | 22 | 201 u/g | 0.16% |
| 20140423-J | 16.0 | 84.0 | 56 | 146 u/g | 0.13% | 20140714-J | 0.8 | 99.2 | 21 | 198 u/g | 0.15% |
| 20140424-J | 18.8 | 81.2 | 59 | 156 u/g | 0.10% | 20140716-J | 0 | 100 | 25 | 226 u/g | 0.17% |
| 20140522-J | 15.3 | 84.7 | 60 | 162 u/g | 0.12 | 20140805-J | 3.2 | 96.8 | 23 | 212 u/g | 0.16% |
| 20140523-J | 21.5 | 78.5 | 61 | 165 u/g | 0.11% | 20140807-J | 1.4 | 98.6 | 24 | 225 u/g | 0.16% |
| 20140526-J | 16.87 | 83.13 | 62 | 166 u/g | 0.12% | 20140809-J | 0.8 | 99.2 | 24 | 226 u/g | 0.17% |

### DESCRIPTION OF THE DRAWINGS

FIG. 1 is a schematic structural diagram of the mycelium culture flask according to the present disclosure;

FIG. 2 is a top view of the A cap of the flask cap of the mycelium culture flask according to the present disclosure;

FIG. 3 is a side view of the A cap of the flask cap of the mycelium culture flask according to the present disclosure;

FIG. 4 is a side view of the B cap of the flask cap of the mycelium culture flask according to the present disclosure;

FIG. 5 is a top view of the inner cap of the flask cap of the mycelium culture flask according to the present disclosure.

In the Figures,
1. flask body;
2. flask cap;
3. A cap;
4. B cap;
5. inner pad;
6. A hole;
7. gap;
8. B hole.

### DETAILED DESCRIPTION OF THE EMBODIMENTS

### Example 1

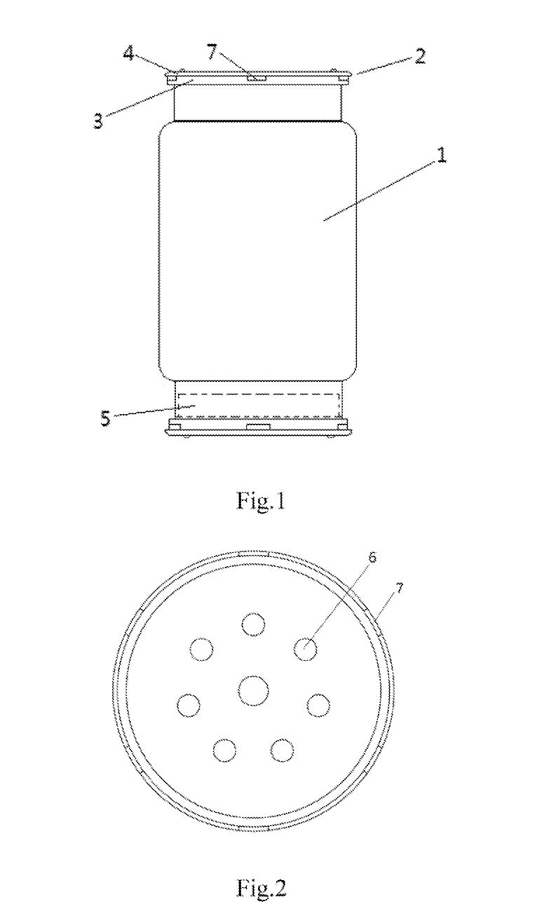

By reference to FIG. 1, the mycelium culture flask comprises a flask body 1 with an upper opening and a lower opening and two flask caps 2 configured to be snap-fitted with the upper opening and the lower opening respectively, and gaps 7 are provided at the edge of the flask cap 2.

### Example 2

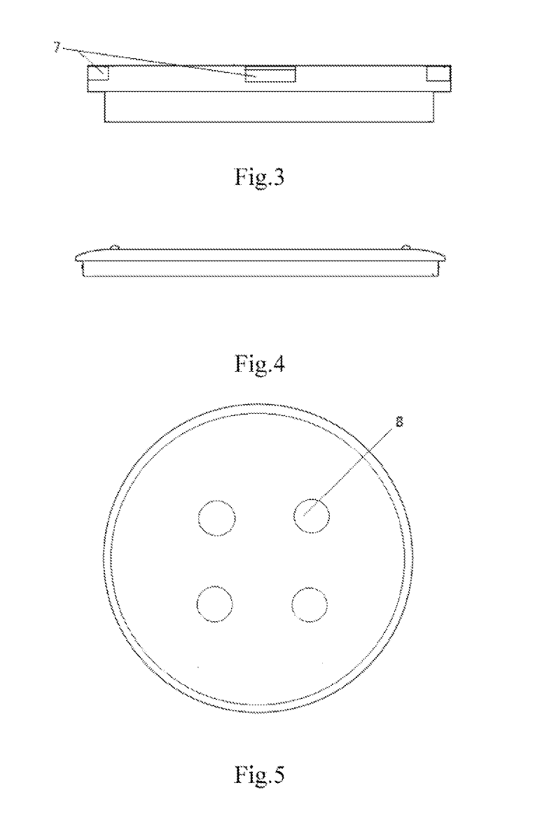

By reference to FIGS. 1 to 5, the mycelium culture flask comprises a flask body 1 with an upper opening and a lower opening and two flask caps 2 configured to be snap-fitted with the upper opening and the lower opening respectively; the flask cap 2 comprises an A cap 3 configured to be snap-fitted with the opening and a B cap 4 configured to be snap-fitted with the A cap 3; a sponge is sandwiched between the A cap 3 and the B cap 4; A holes 6 are provided in a face of the A cap; and gaps 7 are provided at the edge of the A cap 3 in contact with the B cap 4. An inner pad 5 is also provided at a neck of the lower opening of the flask body 1, and B holes 8 are provided in a face of the inner pad 5. The inner pad 5 engages with the inner edge of the lower opening of the flask body 1 through its protruding pad edge.

1. A mycelium culture flask, comprising a flask body with an upper opening and a lower opening and two flask caps snap-fitted with the upper opening and the lower opening respectively, and gaps are provided at the edge of the flask cap.

2. The culture flask according to claim 1, wherein the flask cap comprises an A cap configured to be snap-fitted with the opening and a B cap configured to be snap-fitted with the A cap; a sponge is sandwiched between the A cap and the B cap; A holes are provided in a face of the A cap; and the gap is provided at the edge of the A cap in contact with the B cap.

3. The culture flask according to claim 2, wherein an inner pad is also provided at the neck of the lower opening of the flask body, and B holes are provided in the face of the inner pad.

4. The culture flask according to claim 3, wherein the inner pad engages with the inner edge of the lower opening of the flask body through its protruding pad edge.
